(19) United States
(12) Patent Application Publication
(10) Pub. No.: US 2007/0213703 A1
(43) Pub. Date: Sep. 13, 2007

(54) ELECTRODE FOR RADIO FREQUENCY TISSUE ABLATION

(76) Inventors: Jang Hyun Naam, Seoul (KR); Seung Mun Naam, Seoul (KR)

Correspondence Address: SQUIRE, SANDERS & DEMPSEY L.L.P, PATENT DEPARTMENT, ONE MARITIME PLAZA, SUITE 300, SAN FRANCISCO, CA, 94111-3492, US

(21) Appl. No.: 11/682,317

(22) Filed: Mar. 6, 2007

(30) Foreign Application Priority Data
Mar. 13, 2006 (KR) 10-2006-023023
Mar. 13, 2006 (KR) 10-2006-023024

(51) Int. Cl. A61B 18/14

(52) U.S. Cl. 606/41

(57) ABSTRACT

Provided is an electrode for radio frequency tissue ablation, including: a grip provided with a switch for power control; a hollow electrode connected to one side of the grip, coated with an insulating material, and having an internal space; an electrode needle part provided in one end of the hollow electrode and formed to penetrate tissue; a refrigerant guide pipe inserted into the hollow electrode and supplying/discharging a refrigerant for cooling the electrode needle part and the hollow electrode; and a guide needle externally coupled to the hollow electrode and maintaining the hollow electrode in a straight line by a predetermined length from one side of the hollow electrode.

### CROSS-REFERENCE TO RELATED APPLICATION

This application claims the benefit of Korean Patent Application Nos. 2006-23023 and 2006-23024 which were filed on Mar. 13, 2006, which is hereby incorporated by reference as if fully set forth herein.

### BACKGROUND OF THE INVENTION

1. Field of the Invention

The present invention relates to an electrode for radio frequency tissue ablation, and more particularly, to an electrode for radio frequency tissue ablation, which enables an operator to directly control power and perform an operation while more precisely positioning a radio frequency electrode at a diseased part.

2. Discussion of Related Art

In general, there has been disclosed a medical technology in which an electrode for radio frequency tissue ablation, i.e., a long hollow electrode penetrates into biologic tissue to coagulate or ablate the tissue with radio frequency energy.

When an electric current flows through the tissue, the tissue is heated so that the tissue and a blood vessel are coagulated by a complex biochemical mechanism.

At this time, a cell, which includes the tissue, the blood vessel and blood, is mainly coagulated by thermal modification of protein in the cell at a temperature of about 60° C. or more.

FIG. 1 is a perspective view of a conventional electrode for radio frequency tissue ablation.

As shown in FIG. 1, the electrode for radio frequency tissue ablation includes a grip 110 taking a firm hold at an operation, and a thin and long hollow electrode 122 provided at one side of the grip 110. The hollow electrode 122 is divided into an insulation part 123 having a predetermined length and an electrification part 127 disposed at one side of the insulation part 123. The electrification part 127 has an electrode needle part 126 at the end thereof, and the electrode needle part 126 is typically shaped like a circular cone or a triangular pyramid to easily penetrate the tissue.

Further, a power line 132, a supplying pipe 134 and a discharging pipe 136 are provided at the other side of the grip 110. The power line 132 is used for supplying power to the hollow electrode 122, the supplying pipe 134 is used for supplying a refrigerant so as to control heat generation of the hollow electrode 122, and the discharging pipe 136 is used for discharging the refrigerant after heat exchange.

However, while the electrode needle part 126 of the electrification part 127 penetrates the tissue corresponding to a diseased part and is adjusted to be positioned at the diseased part, such a conventional electrode for radio frequency tissue ablation has a difficulty in precisely positioning the electrification part 127 at the diseased part because resistance due to density of the tissue bends the insulation part 123 provided at one side of the grip 110.

Further, the conventional electrode for radio frequency tissue ablation does not allow an operator to directly control the power of the hollow electrode 122 during surgery. That is, a power switch for the hollow electrode 122 is separately provided from the hollow electrode 122, i.e., placed in an apparatus controller (not shown), so that the operator has to control the power of the hollow electrode 122 wirelessly, by wire or by word of mouth. Accordingly, the power supplied to the electrode for radio frequency tissue ablation is not precisely controlled.

### SUMMARY OF THE INVENTION

The present invention is directed to an electrode for radio frequency tissue ablation, which enables an operator to directly control power and perform an operation while more precisely positioning a radio frequency electrode at a diseased part.

According to an aspect of the invention, an electrode for radio frequency tissue ablation, comprises: a grip provided with a switch for power control; a hollow electrode connected to one side of the grip, coated with an insulating material, and having an internal space; an electrode needle part provided in one end of the hollow electrode and formed to penetrate tissue; a refrigerant guide pipe inserted into the hollow electrode and supplying/discharging a refrigerant for cooling the electrode needle part and the hollow electrode; and a guide needle externally coupled to the hollow electrode and maintaining the hollow electrode in a straight line by a predetermined length from one side of the hollow electrode.

The guide needle may comprise a receiving part that is placed at one end thereof to be contacted and engaged with one side of the grip, and provided as a counter part of an insertion part provided in the one side of the grip.

The guide needle may be hollow to insert the hollow electrode thereinto, and comprise a holder to hold the guide needle at one side thereof.

The guide needle may be detachably coupled to the outside of the hollow electrode, and formed of a steel material to reinforce strength of the hollow electrode.

The guide needle may be formed of a steel material to support the outside of the hollow electrode, and have a predetermined thickness and an inclined surface to be smoothly connected with the hollow electrode.

The diameter of the guide needle may gradually decrease toward a direction connected with the hollow electrode, and the hollow electrode may be bent at a predetermined angle at one end of the guide needle.

The grip may comprise a supplying pipe connected to the refrigerant guide pipe provided in the hollow electrode, and a discharging pipe connected to a space between the hollow electrode and the refrigerant guide pipe. The supplying pipe and the discharging pipe may penetrate the grip.

The refrigerant guide pipe may have a diameter smaller than an inner diameter of the hollow electrode, be inserted into the hollow electrode, introduce a refrigerant for cooling a part of the hollow electrode contacting tissue and the electrode needle part into the hollow electrode, and discharge the refrigerant undergoing heat exchange to the outside of the tissue through the discharging pipe via a space between the refrigerant guide pipe and the hollow electrode.

### BRIEF DESCRIPTION OF THE DRAWINGS

The above and other features and advantages of the present invention will become more apparent to those of ordinary skill in the art by describing in detail preferred embodiments thereof with reference to the attached drawings in which:

FIG. 1 is a perspective view of a conventional electrode for radio frequency tissue ablation;

FIG. 2 is a perspective view of an electrode for radio frequency tissue ablation according to a first exemplary embodiment of the present invention;

FIG. 3 is an exploded perspective view of the electrode according to the first exemplary embodiment of the present invention;

FIG. 4 is an exploded perspective view illustrating an interior structure of the electrode according to the first exemplary embodiment of the present invention;

FIG. 5 is a partial sectional view illustrating a refrigerant flow in the electrode according to the first exemplary embodiment of the present invention;

FIG. 6 is a perspective view of an electrode for radio frequency tissue ablation according to a second exemplary embodiment of the present invention;

FIG. 7 is a perspective view illustrating an interior structure of the electrode according to the second exemplary embodiment of the present invention; and

FIG. 8 is a perspective view of an electrode for radio frequency tissue ablation according to a third exemplary embodiment of the present invention.

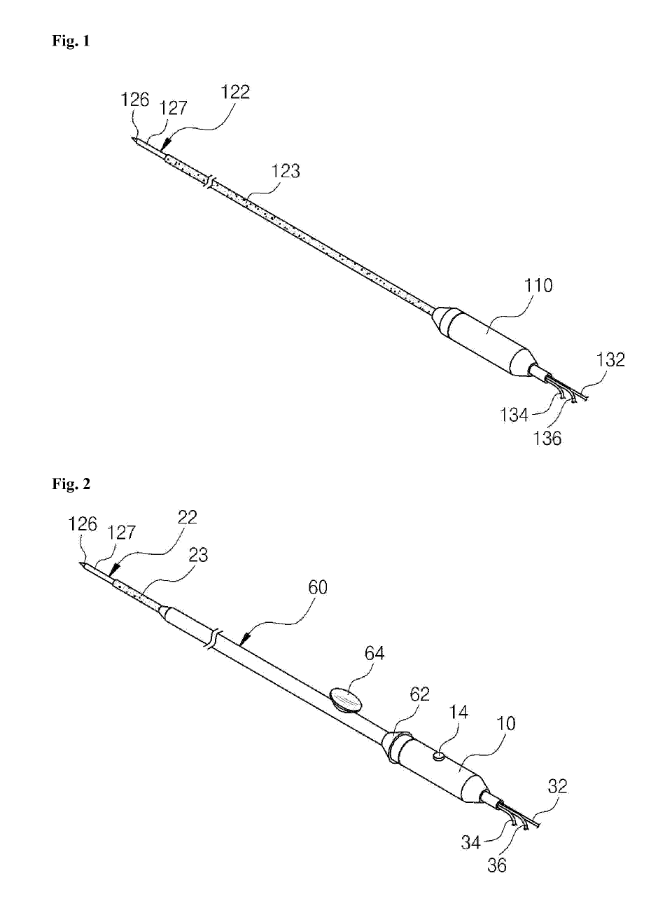

### DETAILED DESCRIPTION OF EXEMPLARY EMBODIMENTS

Hereinafter, exemplary embodiments of the present invention will be described in detail with reference to the accompanying drawings, in which like numerals refer to like elements and repetitive descriptions will be avoided as necessary.

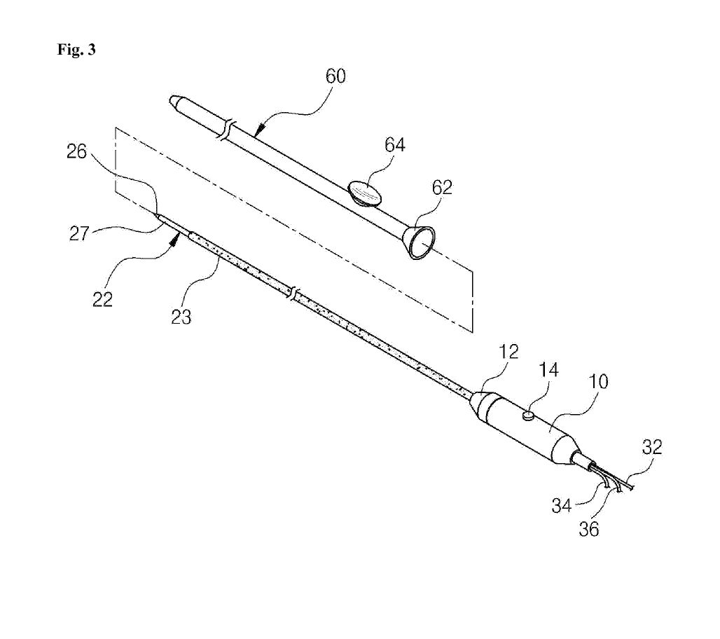

FIGS. 2 and 3 are a perspective view and an exploded perspective view of an electrode for radio frequency tissue ablation according to a first exemplary embodiment of the present invention.

As shown in FIGS. 2 and 3, the electrode for radio frequency tissue ablation includes a grip 10, a hollow electrode 22, and a guide needle 60.

The hollow electrode 22 is connected to one side of the grip 10, and includes an electrode needle part 26 having a pointed tip. Further, a switch 14 is provided on an outer surface of the grip 10 so as to control power of the electrode for radio frequency tissue ablation.

The switch 14 is used to control the power of the electrode for radio frequency tissue ablation. It is preferable but not necessary that the switch 14 is provided in a sliding or dial type enabling a stepwise power control like a power button of a general vacuum cleaner. Alternatively, the switch may be provided in a button type.

Further, the hollow electrode 22 connected to one side of the grip 10 is divided into an insulation part 23 provided by a predetermined length from the grip 10 and an electrification part 27 provided at one end of the insulation part 23

The electrode needle part 26 has a pointed tip enough to penetrate tissue. Here, the pointed tip may be shaped like a circular cone or a triangular pyramid.

Meanwhile, the guide needle 60 includes a receiving part 62 to receive an insertion part 12 provided in one side of the grip 10, so that the guide needle 60 can be detachably contacted and engaged with the insertion part 12. Accordingly, the insertion part 12 and the receiving part 62 cause the guide needle 60 to be firmly supported in the grip 10.

Thus, the hollow electrode 22 is connected to one side of the grip 10 and inserted inside the guide needle 60 while the guide needle 60 is closely contacted and engaged with one side of the grip 10. Additionally, a power line 32, a supplying pipe 34 and a discharging pipe 36 are provided at the other side of the grip 10. The power line 32 is used for supplying power to the hollow electrode 22, the supplying pipe 34 is used for supplying a refrigerant so as to control temperature of the hollow electrode 22, and the discharging pipe 36 is used for discharging the refrigerant after heat exchange.

Here, the supplying pipe 34 and the discharging pipe 36 may penetrate the grip 10.

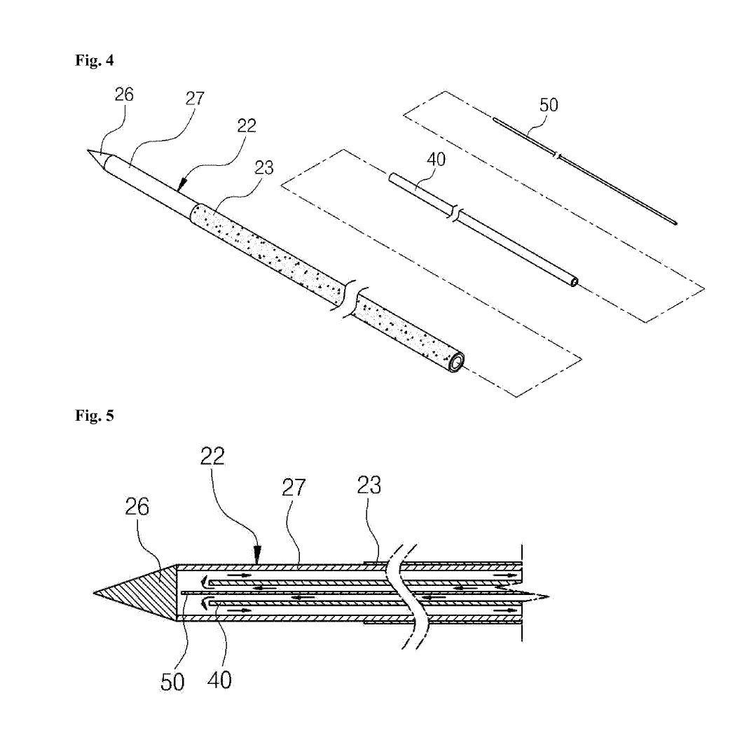

FIG. 4 is an exploded perspective view illustrating an interior structure of the electrode according to the first exemplary embodiment of the present invention.

As shown in FIG. 4, the electrode for radio frequency tissue ablation according to the first exemplary embodiment includes a refrigerant guide pipe 40 inserted into the hollow electrode 22 which includes the electrode needle part 26, the electrification part 27 and the insulation part 23; and a temperature sensor line 50 inserted into the refrigerant guide pipe 40.

Here, the refrigerant guide pipe 40 is filled with a refrigerant so as to control heat generation of the electrification part 27 provided in the hollow electrode 22 according as the electrode for radio frequency tissue ablation is powered on, and the temperature sensor line 50 may be inserted into the refrigerant guide pipe 40.

Also, the temperature sensor line 50 is inserted into the refrigerant guide pipe 40 and extends toward a predetermined inner position of the electrification part 27, so that it senses the temperature of the electrification part 27, thereby enabling a controller (not shown) for controlling the power of the electrode for radio frequency tissue ablation to determine the time to control the power.

FIG. 5 is a partial sectional view illustrating a refrigerant flow in the electrode according to the first exemplary embodiment of the present invention.

Referring to FIG. 5, in the refrigerant flow in the electrode for radio frequency tissue ablation according to the first embodiment of the present invention, the hollow electrode 22 internally includes the refrigerant pipe 40 through which the refrigerant flows, and the temperature sensor line 50 inside the refrigerant pipe 40 to sense the temperature of the electrification part 27.

Here, the refrigerant for controlling the heat generation of the electrification part 27 provided at one side of the hollow electrode 22 is supplied along a space between the temperature sensor line 50 and the refrigerant pipe 40 and introduced into the electrification part 27. After heat exchange, the refrigerant is discharged along a space between an inner wall of the hollow electrode 22 and an outer wall of the refrigerant pipe 40.

As shown in FIGS. 2 and 3, it is preferable but not necessary that the refrigerant flow circulates through the supplying pipe 34 and the discharging pipe 36 which are connected to one side of the grip 10.

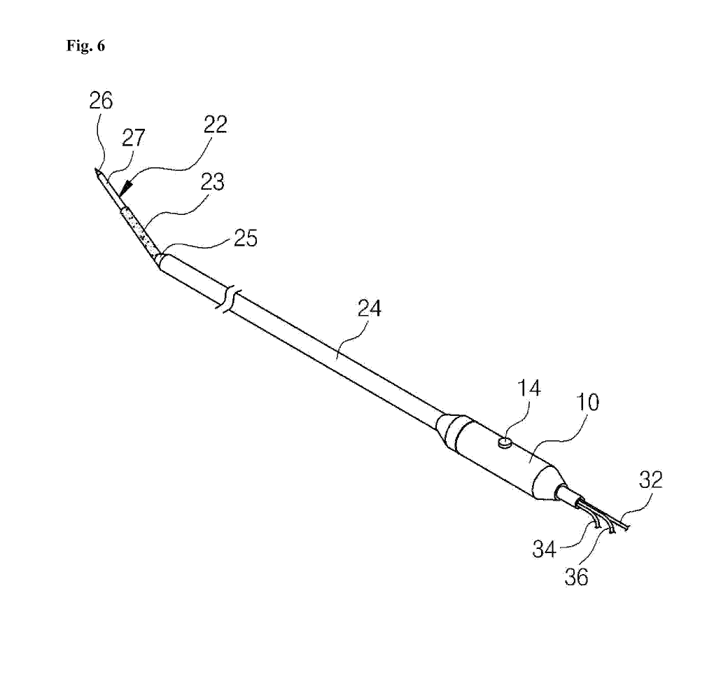

FIG. 6 is a perspective view of an electrode for radio frequency tissue ablation according to a second exemplary embodiment of the present invention.

As shown in FIG. 6, the electrode for radio frequency tissue ablation according to the second exemplary embodiment of the present invention includes a grip 10, a guide needle 24, and a hollow electrode 22.

Here, the grip 10 is provided with a switch 14 on a predetermined outer position thereof, and connected with a guide needle 24 at one side thereof. Here, the guide needle 24 and the hollow electrode 22 are formed as a single body. The guide needle 24 is provided with an inclined surface 25 and connected to an insulation part 23 of the hollow electrode 22. Additionally, an electrification part 27 and an electrode needle part 26 are in turn disposed in one side of the insulation part 23.

At this time, the switch 14 is employed to control the power of the electrode for radio frequency tissue ablation. It is preferable but not necessary that the switch 14 is provided in a sliding or dial type enabling a stepwise power control like a power button of a general vacuum cleaner. Alternatively, the switch may be provided in a button type.

Further, the electrode needle part 26 has a tapered tip enough to penetrate tissue. Here, the tapered tip may be shaped like a circular cone or a triangular pyramid.

Meanwhile, the hollow electrode 22 includes the electrode needle part 26, the electrification part 27 following the electrode needle part 26, and the insulation part 23 following the electrification part 27, and a part provided at one side of the guide needle 24 and connected to the inclined surface 25 is bent at a predetermined angle.

It is preferable but not necessary that the angle ranges from 0° C. to 45° C. so that the electrification part 27 of the hollow electrode 22 can be more precisely positioned at a diseased part of the sick.

Thus, the hollow electrode 22 and the guide needle 24, which is integrally provided with the insulation part 23, are connected to one side of the grip 10. Additionally, a power line 32, a supplying pipe 34 and a discharging pipe 36 are provided at the other side of the grip 10. The power line 32 is used for supplying power to the hollow electrode 22, the supplying pipe 34 is used for supplying a refrigerant so as to control temperature of the hollow electrode 22, and the discharging pipe 36 is used for discharging the refrigerant after heat exchange.

Here, the supplying pipe 34 and the discharging pipe 36 may penetrate the grip 10.

Using the foregoing electrode for radio frequency tissue ablation, an operation order is as follows: the electrode needle part 26, the electrification part 27 and the insulation part 23 are sequentially inserted into the tissue, and then the guide needle 24 is smoothly inserted by the inclined surface 25 provided at one side of the guide needle 24 while positioning the electrification part 27 at the diseased part. After the electrification part 27 is precisely positioned at the diseased part, the power is supplied to the electrification part 27, so that the electrification part 27 is heated to thereby cure the diseased part.

At this time, the guide needle 24 allows the electrification part 27 to be precisely positioned at the diseased part irrespective of resistance due to density of the tissue. Because the guide needle 24 has a bending angle of α, it can be more precisely positioned at the diseased part. Further, the operator can directly control power through the switch 14 provided in the grip 10, thereby achieving a more precise operation.

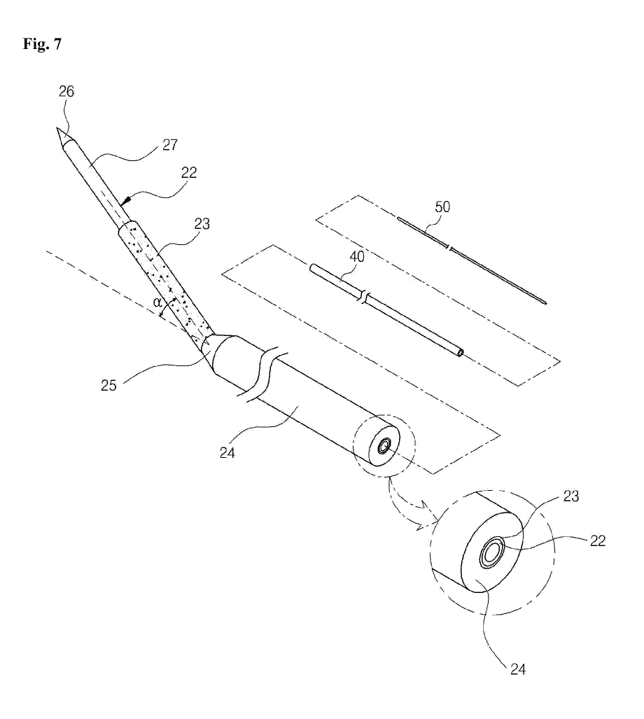

FIG. 7 is a perspective view illustrating an interior structure of the electrode according to the second exemplary embodiment of the present invention.

As shown in FIG. 7, the electrode for radio frequency tissue ablation according to the second exemplary embodiment of the present invention includes a refrigerant guide pipe 40 inserted into the hollow electrode 22 which includes the electrode needle part 26, the electrification part 27 and the insulation part 23; and a temperature sensor line 50 inserted into the refrigerant guide pipe 40.

Here, the refrigerant guide pipe 40 is filled with a refrigerant so as to control heat generation of the electrification part 27 provided in the hollow electrode 22 according as the electrode for radio frequency tissue ablation is powered on, and the temperature sensor line 50 is inserted into the refrigerant guide pipe 40.

Also, the temperature sensor line 50 is inserted into the refrigerant guide pipe 40 and extends toward a predetermined inner position of the electrification part 27, so that it senses the temperature of the electrification part 27, thereby enabling a controller (not shown) for controlling the power of the electrode for radio frequency tissue ablation to determine the time to control the power.

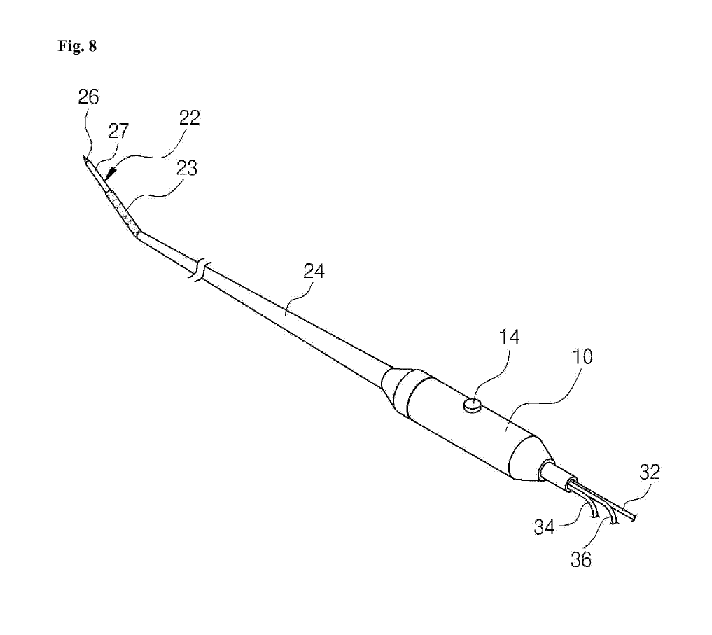

FIG. 8 is a perspective view of an electrode for radio frequency tissue ablation according to a third exemplary embodiment of the present invention.

As shown in FIG. 8, the electrode for radio frequency tissue ablation according to the third exemplary embodiment of the present invention includes a grip 10, a guide needle 24 and a hollow electrode 22.

Here, the grip 10 is provided with a switch 14 on a predetermined outer position thereof, and connected with the guide needle 24 at one side thereof. Here, the guide needle 24 and the hollow electrode 22 are formed as a single body. The guide needle 24 is connected to an insulation part 23 of the hollow electrode 22. Additionally, an electrification part 27 and an electrode needle part 26 are in turn disposed in one side of the insulation part 23.

Further, the electrode needle part 26 has a tapered tip enough to penetrate tissue. Here, the tapered tip may be shaped like a circular cone or a triangular pyramid.

The diameter of the guide needle 24 is the same as that of the insulation part 23 at a predetermined position, and gradually increases as going toward the grip 10.

Meanwhile, the hollow electrode 22 includes the electrode needle part 26, the electrification part 27 following the electrode needle part 26, and the insulation part 23 following the electrification part 27, and the hollow electrode 22 is bent between the insulation part 23 and the guide needle 24 at a predetermined angle.

It is preferable but not necessary that the angle ranges from 0° C. to 45° C. so that the electrification part 27 of the hollow electrode 22 can be more precisely positioned at a diseased part of the sick.

Thus, the hollow electrode 22 and the guide needle 24, which is integrally provided with the insulation part 23, are connected to one side of the grip 10. Additionally, a power line 32, a supplying pipe 34 and a discharging pipe 36 are provided at the other side of the grip 10. The power line 32 is used for supplying power to the hollow electrode 22, the supplying pipe 34 is used for supplying a refrigerant so as to control temperature of the hollow electrode 22, and the discharging pipe 36 is used for discharging the refrigerant after heat exchange.

Here, the supplying pipe 34 and the discharging pipe 36 may penetrate the grip 10.

Using the foregoing electrode for radio frequency tissue ablation, an operation order is as follows: the electrode needle part 26, the electrification part 27 and the insulation part 23 are sequentially inserted into the tissue, and then the guide needle 24 is smoothly inserted by the same diameter as the insulation part 23 while positioning the electrification part 27 at the diseased part. After the electrification part 27 is precisely positioned at the diseased part, the power is supplied to the electrification part 27, so that the electrification part 27 is heated to thereby cure the diseased part.

At this time, the guide needle 24 allows the electrification part 27 to be precisely positioned at the diseased part irrespective of resistance due to density of the tissue. Because the guide needle 24 has a bending angle of α, it can be more precisely positioned at the diseased part. Further, the operator can directly control power through the switch 14 provided in the grip 10, thereby achieving a more precise operation.

As described above, the electrode for radio frequency tissue ablation has the following effects.

First, the guide needle is provided to reinforce the strength of the insulation part of the hollow electrode, thereby precisely positioning the electrification part at a diseased part irrespective of the resistance due to the density of the tissue.

Second, the switch is provided in the grip so that an operator can directly control power during surgery using the electrode for radio frequency tissue ablation, thereby precisely controlling the heat generation of the electrification part.

Third, the guide needle is detachably provided so that it can be readily replaced with another guide needle having a different length as necessary.

Fourth, the guide needle is provided with a receiving part at one side thereof to receive an insertion part provided in one side of the grip, so that the guide needle can be detachably contacted and engaged with the insertion part, thereby firmly supporting the guide needle to the grip.

Fifth, the electrification part generating heat is bent at a predetermined angle, thereby more precisely positioning the electrode for radio frequency tissue ablation at a diseased part.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. An electrode for radio frequency tissue ablation, comprising:
a grip provided with a switch for power control;
a hollow electrode connected to one side of the grip, coated with an insulating material, and having an internal space;
an electrode needle part provided in one end of the hollow electrode and formed to penetrate tissue;
a refrigerant guide pipe inserted into the hollow electrode and supplying/discharging a refrigerant for cooling the electrode needle part and the hollow electrode; and
a guide needle externally coupled to the hollow electrode and maintaining the hollow electrode in a straight line by a predetermined length from one side of the hollow electrode.

2. The electrode for radio frequency tissue ablation according to claim 1, wherein the guide needle comprises a receiving part that is placed at one end thereof to be contacted and engaged with one side of the grip, and provided as a counter part of an insertion part provided in the one side of the grip.

3. The electrode for radio frequency tissue ablation according to claim 1, wherein the guide needle is hollow to insert the hollow electrode thereinto, and comprises a holder to hold the guide needle at one side thereof.

4. The electrode for radio frequency tissue ablation according to claim 1, wherein the guide needle is detachably coupled to the outside of the hollow electrode, and formed of a steel material to reinforce strength of the hollow electrode.

5. The electrode for radio frequency tissue ablation according to claim 1, wherein the guide needle is formed of a steel material to support the outside of the hollow electrode, and has a predetermined thickness and an inclined surface to be smoothly connected to the hollow electrode.

6. The electrode for radio frequency tissue ablation according to claim 5, wherein the hollow electrode is bent at a predetermined angle at one end of the guide needle.

7. The electrode for radio frequency tissue ablation according to claim 1, wherein a diameter of the guide needle gradually decreases toward a direction connected with the hollow electrode.

8. The electrode for radio frequency tissue ablation according to claim 7, wherein the hollow electrode is bent at a predetermined angle at one end of the guide needle.

9. The electrode for radio frequency tissue ablation according to claim 1, wherein the grip comprises a supplying pipe connected to the refrigerant guide pipe provided in the hollow electrode, and a discharging pipe connected to a space between the hollow electrode and the refrigerant guide pipe, the supplying pipe and the discharging pipe penetrating the grip.

10. The electrode for radio frequency tissue ablation according to claim 9, wherein the refrigerant guide pipe has a diameter smaller than an inner diameter of the hollow electrode, is inserted into the hollow electrode, introduces a refrigerant for cooling a part of the hollow electrode contacting the tissue and the electrode needle part into the hollow electrode, and discharges the refrigerant undergoing heat exchange to the outside of the tissue through the discharging pipe via a space between the refrigerant guide pipe and the hollow electrode.
